(19) United States
(12) Patent Application Publication
(10) Pub. No.: US 2025/0266142 A1
(43) Pub. Date: Aug. 21, 2025

(54) MEDICATION ADHERENCE PLATFORM FOR MEDICATION DETECTION AND DISCERNMENT

(71) Applicant: MediSensor Technologies Inc, Palo Alto, CA (US)

(72) Inventors: David E. BORISON, Palo Alto, CA (US); Nicholas A. KNUDSON, Highwood, IL (US)

(21) Appl. No.: 18/443,795

(22) Filed: Feb. 16, 2024

(51) Int. Cl. G16H 20/10; A61J 1/03; A61J 7/02

(52) U.S. Cl. CPC G16H 20/10; A61J 1/03; A61J 7/02

(57) ABSTRACT

A medication adherence platform that includes a pill case platform having a pill case container, a load cell, and circuitry that determines a weight of the pill case container, and a server that determines whether the pill case container contains specified quantities of one or more pill types based on the sensed weight, a patient medication schedule, and a database of pill weights. The server may determine a corrective quantity of one or more pill types and/or whether the pill case container contains an incorrect pill type. The database may include multiple pill weights for a pill type (e.g., crowd-sourced pill weights), and the server may evaluate the weight based further on a statistical analysis of the multiple pill weights. The server may provide alerts to the pill case platform and/or instructions/alerts/pill images to a user device. The pill case platform may include multiple pill case containers (e.g., 1/day).

### TECHNICAL FIELD

Examples of the present disclosure generally relate to medication devices and, more particularly, to a medication adherence platform for medication detection and discernment.

### BACKGROUND

It has been reported that over 160 million Americans suffer from chronic diseases, more than 50% of whom report confusion regarding when, how, and why to take prescription medications. Failure to take prescription medications reportedly costs the U.S. economy hundreds of billions of dollars per year, results in over 100,000 preventable deaths per year, is responsible for 25% of hospital admissions, and 22% of nursing home admissions.

Medication containers (i.e., “pill cases”) with compartments for storing daily dosages of medications for a patient are not suitable for patients with cognitive decline, physical/motor disabilities, visual disabilities, and/or other disabilities. For such patients, it may be challenging to identify/load the correct number and types of medications in the appropriate compartments, and to remember which medications to take and when to take them.

### SUMMARY

Medication adherence platforms for medication detection and discernment are described herein. One example is a system that includes a pill case platform having a pill case container, a load cell, and circuitry that determines a container weight of the pill case container based on an output of the load cell. The system further includes a host device that determines whether the pill case container contains specified quantities of multiple pill types of a patient medication schedule based on the container weight and a database of pill types and associated pill weights.

Another example described herein is a non-transitory computer readable medium encoded with a computer program that includes instructions to cause a processor to associate a pill case platform with a patient and with a user device, where the pill container platform includes multiple pill case containers, receive a medication schedule of the patient from the user device, determine specified quantities of multiple pill types of the patient medication schedule, for each of the pill case containers, based on the patient medication schedule, receive indications of container weights from the pill case platform, and determine whether the pill case containers contain the specified quantities of the multiple pill types of the patient medication schedule based on the container weights and a database of pill weights and associated pill types.

Another example described herein is a method that includes associating a pill case platform with a patient and with a user device, where the pill container platform a pill case container, a load cell, and circuitry that determines a container weight of the pill case container based on an output of the load cell, and determining whether the pill case container contains specified quantities of multiple pill types of a patient medication schedule based on the container weight and a database of pill types and associated pill weights.

### BRIEF DESCRIPTION OF DRAWINGS

So that the manner in which the above recited features can be understood in detail, a more particular description, briefly summarized above, may be had by reference to example implementations, some of which are illustrated in the appended drawings. It is to be noted, however, that the appended drawings illustrate only typical example implementations and are therefore not to be considered limiting of its scope.

FIG. 1 is a block diagram of a medication adherence platform, according to an embodiment.

FIG. 2 is a block diagram of pill case platform of the medication adherence platform, according to an embodiment.

FIG. 3 illustrates a method of ensuring patient adherence to a medication schedule, according to an embodiment.

FIG. 4 illustrates a method of determining weight detection events of a pill case container, according to an embodiment.

FIG. 5 illustrates a method of pre-processing sensed load data, according to an embodiment.

FIG. 6 is a block diagram of a host computer system, according to an embodiment.

To facilitate understanding, identical reference numerals have been used, where possible, to designate identical elements that are common to the figures. It is contemplated that elements of one example may be beneficially incorporated in other examples.

### DETAILED DESCRIPTION

Various features are described hereinafter with reference to the figures. It should be noted that the figures may or may not be drawn to scale and that the elements of similar structures or functions are represented by like reference numerals throughout the figures. It should be noted that the figures are only intended to facilitate the description of the features. They are not intended as an exhaustive description of the features or as a limitation on the scope of the claims. In addition, an illustrated example need not have all the aspects or advantages shown. An aspect or an advantage described in conjunction with a particular example is not necessarily limited to that example and can be practiced in any other examples even if not so illustrated, or if not so explicitly described.

Embodiments herein describe a medication adherence platform for medication detection and discernment. The medication adherence platform may be useful to ensure that users (i.e., patients and/or care providers) load appropriate numbers of appropriate pill types in pill cases, and remove/dispense appropriate numbers of appropriate pill types at appropriate times.

As used herein, the term “pill type” refers to a pill of a medication type (e.g., acetaminophen), and a medication dosage. The term “medication dosage” refers to a weight of the medication type (e.g., 500 mg of acetaminophen). The term “pill weight” refers to the actual weight of the pill, which includes the weight of the medication (i.e., medication dosage), plus the weight of any fillers (i.e., additional ingredients that add weight to a pill type). The pill weight does not necessarily reflect the medication dosage of a pill. As an example, and without limitation, a pill containing 500 mg of acetaminophen may have a pill weight of approximately 1335 mg. The medication type and medication dosage may be represented by a National Drug Code or Universal Product Code.

In an example, a medication adherence platform includes one or more patient-specific pill case platforms for holding medications of a patient, an application program for respective user devices (e.g., mobile telephones, tablets, personal computers, and/or other smart devices), a host (e.g., a server) that provides back-end services, including pill detection and classification, pill weight database management, and an alert management system. The pill case platforms and respective user devices may communicate with one another and/or with the host over wireless communication channels.

The pill case platform includes one or more pill case containers (e.g., one for each day of the week) and one or more load cells (e.g., a load cell for per pill case container and/or a load cell for multiple pill case containers and corresponding circuitry/code to associate weight detection events with respective pill case containers). The pill case platform further includes circuitry that determines container weights and reports the container weights to the host. The circuitry may perform one or more other functions, such as, noise reduction, filtering, and/or adaptive taring (e.g., to compensate for load cell drift).

The host may provide instructions to the application to guide the user through loading and unloading the pill case containers. The host may include an aided loading mode, an aided unloading mode, an unaided loading mode, and/or an unaided unloading mode. In the aided loading and unloading modes, the host provides detailed instructions. The host may provide instructions for each pill case container, and may include instructions for each of multiple pill types to be loaded into, or unloaded from, one or more of the pill case containers. The instructions may include images of the corresponding pills types. The host also determines whether the specified quantities of the specified pill types are properly loaded/unloaded, as the user loads or unloads the pill cases. Upon detection of an error, the host may determine the pill type and quantity that has been improperly loaded or unloaded based on the medication schedule and the database of pill weights, and may provide corrective instructions to the user. The corrective instructions may include text messages, push notifications, audio alarms, LED indicators, and/or images of affected pill types.

In the un-aided loading and unloading modes, the user loads and/or unloads each container of the pill case platform with little or no guidance from the host. Upon completion of unaided loading or unloading, the host determines if the pill case container contains the specified quantities of the specified pill types, based on the patient medication schedule and the database of pill weights. Upon detection of an incorrect weight of a pill case container, the host may determine which pill(s) are improperly loaded or unloaded, and may provide corrective instructions to the user. The corrective instructions may include text messages, push notifications, audio alarms, LED indicators, and/or images of affected pill types.

The host may discern amongst multiple pill types within a pill case container. The host may, for example, instruct the user to load or unload various quantities of multiple pill types in a given pill case container (e.g., designated Tuesday), and may determine whether the specified quantities of the specified pill types have been loaded or unloaded, based on the container weights. If the host determines that the container weights do not correspond to an expected weight, the host may further determine which pill type(s), and quantities, are incorrectly loaded or unloaded. The host may further provide corrective instructions to the user.

The pill weight of a pill type may vary due to one or more of a variety of factors such as, without limitation variations/changes in pill manufacturing, formulations, pill handling, environmental conditions, hardware, user characteristics, and/or other factors. In addition, sensed/detected weights for a pill type may vary amongst pill case platforms due to environmental and/or hardware-based variations. The host may capture variations in pill weights by populating and/or update the database with pill weights obtained from one or more of a variety of sources, including pill weights determined from prior container weights of the pill case platform and/or other pill case platforms (i.e., crowd-sourced pill weights). The host may further perform adaptive classification to account for the variations in pill weights. Adaptive pill classification may provide a tolerance (e.g., a weight range) for a pill type, which may be based on a median weight and a standard deviation amongst pills of the pill type.

The host may maintain multiple databases of pill types and pill weights that are specific to pill case platforms, pill case containers, users, geographic regions, and/or other factors/features, and may tailor pill classification methods/heuristics for the respective databases.

The medication adherence platform may process, store, and transmit medication information in compliance with government statutes/rules/regulations (e.g., the Health Insurance Portability and Accountability Act, or HIPAA).

The medication adherence platform may be useful to ensure that users (i.e., patients and/or care providers) load the appropriate pills in the pill case containers, and remove/dispense the appropriate pills at appropriate times.

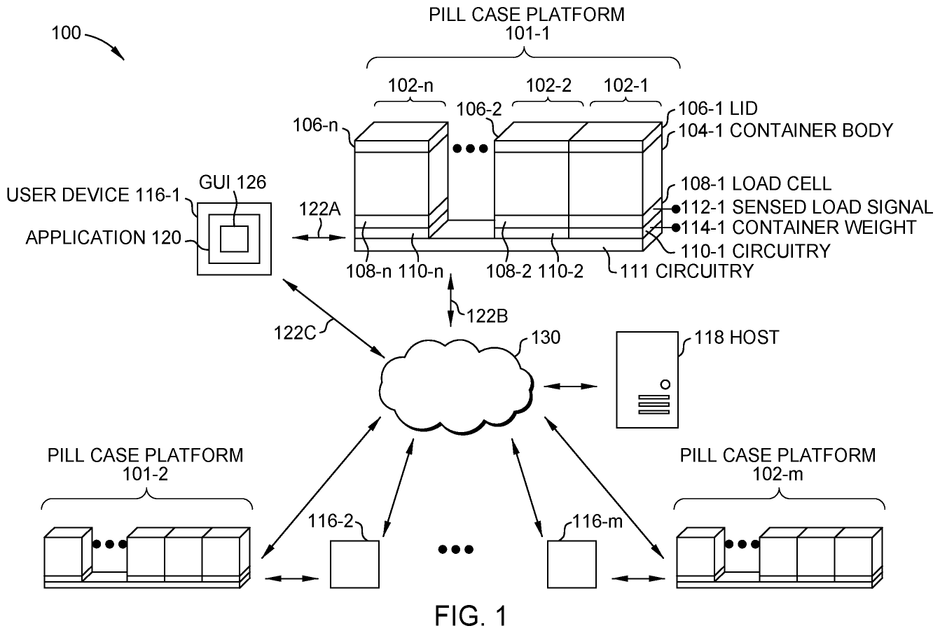

FIG. 1 is a block diagram of a medication adherence platform 100, according to an embodiment. Medication adherence platform 100 includes one or more pill case platforms, illustrated here as pill case platforms 101-1 through 101-m (collectively, pill case platforms 101), corresponding user devices 116-1 through 116-m (collectively, user devices 116), and a host 118 (e.g., a server computer system). Pill case platforms 101-1 through 101-m, or a portion thereof, may be associated with m respective patients, where m is a positive integer. Medication adherence platform 100 may represent a network of pill case platforms 101.

Pill case platform 101-1 includes one or more pill case containers 102-1 through 102-n (collectively, pill case containers 102), where n is a positive integer. In an example, n equals 7 (e.g., one pill case container for each day of the week). Pill case platform 101-1 is not, however, limited to n=7.

Pill case container 102-1 includes a container body 104-1 and a lid 106-1 that secures an opening of container body 104-1. Pill case container 102-1 further includes a load cell 108-1 that senses a load (e.g., a weight of a medication, such as tablets or pills) placed within container body 104-1, and outputs a corresponding sensed load signal 112-1. Pill case container 102-1 may further include a removable tray positioned within container body 104-1, dimensioned to receive medication pills. The tray may rest upon load cell 108-1. Load cell 108-1 may be a relatively inexpensive off-the-shelf load cell, and may be selected and/or optimized/customized for a target weight range and/or other criteria. Load cell 108-1 may output sensed load signal 112-1 as an analog signal.

Pill case container 102-1 further includes circuitry 110-1 that processes sensed load signal 112-1 and outputs container weights associated with loading and/or unloading of pills from pill case container 102-1. In FIG. 1, the container weights are represented as weight detection events 114-1. Weight detection events 114-1 may include a measure of weight (e.g., a mean weight, a container weight, and/or a standard deviation) and/or other information, examples of which are provided further below. Circuitry 110-1 may perform one or more of other functions, such as, without limitation, noise reduction, filtering, weight detection, and/or adaptive taring, examples of which are provided further below. Circuitry 110 may include, without limitation, logic gates, a state machine, and/or a processor/controller and memory (e.g., firmware) encoded with instructions (i.e., code) that are executed by the processor/controller. Circuitry 110 may include a printed circuit board (PCB). Routing and/or a layout of the PCB may be optimized to minimize noise and crosstalk. Circuitry 110 may include a firmware-based state machine.

Pill case platform 101-1 may further include one or more signaling devices, such as light-emitting diodes (LEDs), buzzer, and/or a speaker. Pill case platform 101-1 may, for example, include, an external LED (e.g., a red LED for alert purposes) and/or an internal LED (e.g., on inner surfaces of lids 106 and/or other location(s) within pill case platform 101-1).

User device 116-1 may include a hand-held user device (e.g., a mobile telephone), a stationary user device (e.g., a desk-top computer), and/or other user device. User device 116-1 may include an application 120 (i.e., a computer program or code) that interfaces with pill case platform 101-1 and/or host 118. Application 120 may present a graphical user interface (GUI) 126 on a display of user device 116-1.

Host 118 performs pill detection and classification functions based on weight detection events 114-1. Host 118 may also interface with user device 116-1 and/or pill case platform 101-1. Host 118 may, for example, provide pill loading instructions to user device 116-1 (e.g., aided loading and/or unloading instructions and/or corrective instructions), and/or may provide notifications/alerts to user device 116-1 and/or pill case platform 101-1. Host 118 may also maintain/manage one or more databases of pill types (i.e., medication types and corresponding pill weights), examples of which are provided further below.

Pill case platform 101-1, user device 116-1, and host 118 may include respective communication circuitry to communicate with one another (directly and/or indirectly) over one or more communication links or channels, which may include a wired and/or a wireless communication channel. A wireless communication channel may include, without limitation, a short-range wireless channel, a near-field communication (NFC) channel, a wireless access point to the Internet, and/or other communication channel(s). In the example of FIG. 1, pill case platform 101-1 further includes circuitry 111, which may support (i.e., may be shared/amongst/used by) circuitry 110 of pill case containers 102-1 through 102-n. Circuitry 111 may include communication circuitry that communicates with user device 116-1 over a communication channel 122A and/or with host 118 over a communication channel 122B. User device 116-1 and host 118 may communicate with one another over a communication channel 122C. Communication channel 122B and/or 122C may include a packet-switched communication network 130 (e.g., the Internet).

In another embodiment, load cell 108-1 is shared amongst multiple pill case containers 102, and circuitry 111 includes circuit that associates weight detection events with respective ones of the pill case containers.

Pill case platforms 101-2 through 102-m may be similar to, and/or identical to pill case platform 101-1. In an example, pill case platforms 101-1 through 102-m have the same number of pill case containers, n. In another example, n may differ amongst pill case platforms 101-1 through 102-m.

One or more functions described herein with respect to circuitry 110-1 and/or circuitry 111 may be performed by application 120 and/or host 118. Similarly, one or more functions described herein with respect to host 118, or portions thereof, may be performed by pill case platforms 101 and/or user devices 116.

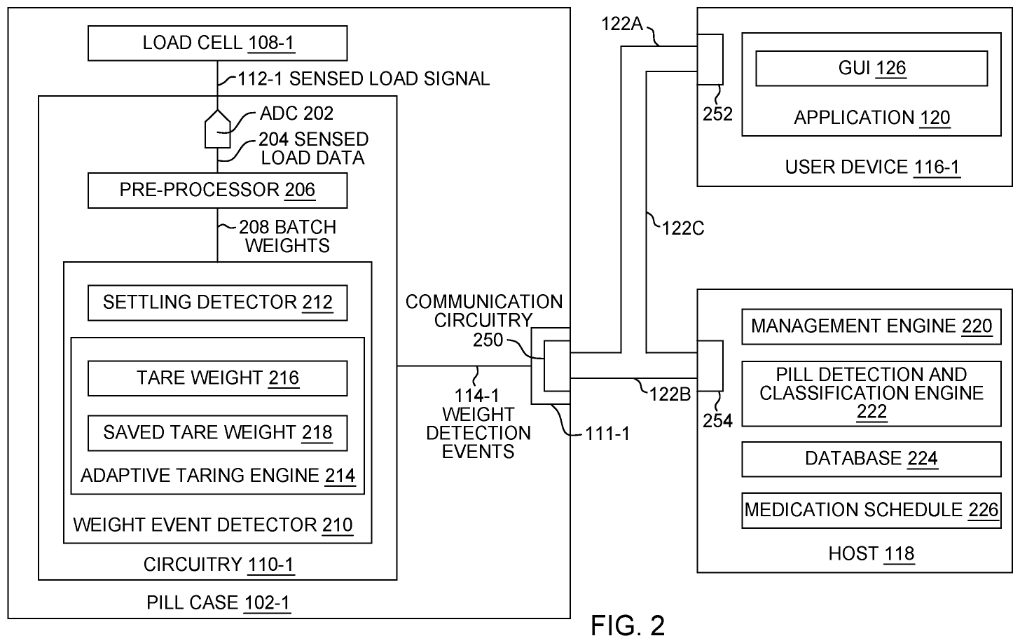

FIG. 2 is a block diagram of pill case platform 101-1, according to an embodiment. In the example of FIG. 2, circuitry 111 includes communication circuitry 250, and user device 116 and host 118 include corresponding communication circuitry 252 and 254, to communicate with one another over corresponding communication channels 122A, 122B, and 122C. Communication circuitry 250, 252, and 254 may include wireless transmitters and/or receivers (e.g., wireless transceivers).

In FIG. 2, circuitry 110-1 includes an analog-to-digital converter (ADC) 202 that converts sensed load signal 112-1 to digital sensed load data 204. Sensed load data 204 may include samples (e.g., of amplitudes) of sensed load signal 112-1. ADC 202 may sample sensed load signal 112-1 at a constant rate (e.g., 80 Hertz). ADC 202 may include relatively inexpensive off-the-shelf ADC circuitry.

Circuitry 110-1 further includes a pre-processor 206 that performs initial weight detection functions. Pre-processor 206 may pre-process batches of samples of sensed load data 204, and output corresponding batch weights 208, such as described further below. Pre-processor 206 may perform other functions such as, without limitation, noise reduction and/or filtering.

Circuitry 110 further includes a weight event detector 210 that monitors batch weights 208 for weight detection events 114-1. Weight event detector 210 may monitor batch weights 208 when lid 106-1 is open. Weight event detector 210 may detect a weight detection event 114-1 when batch weights 208 exceed a tare weight 216. Weight event detector 210 may include a settling detector 212 that determines whether batch weights 208 that exceed tare weight 216 have sufficiently settled to be deemed reliable, such as described further below. Weight event detector 210 may further include an adaptive taring engine 214 that dynamically adapts a tare weight 216, such as described further below.

In FIG. 2, host 118 includes a management engine 220, a pill detection and classification engine 222, a database 224 of pill types and associated pill weights, and a patient medication schedule 226 associated with pill case platform 101-1. Patient medication schedule 226 may include listing of one or more pill types (e.g., medication types and medication weight/dosage), and corresponding patient dosages (e.g., a daily and/or hourly dosage schedule).

Management engine 220 may interface with user device 116-1 over communication channel 122C to receive patient medication schedule 226, provisioning parameters, and/or configuration parameters, and/or may send messages (e.g., instructions, prompts, and/or alerts) to user device 116-1. Management engine 220 may send instructions to user device 116-1 for presentation in GUI 126, and/or may receive user input via GUI 126.

Management engine 220 may interface with pill case platform 101-1 over communication channel 122B. Management engine 220 may, for example, send prompts and/or alerts a signaling device (e.g., an LED, a buzzer, and/or a speaker) of pill case platform 101-1, to alert a user of a condition and/or to prompt the user to perform an action. Management engine 220 may prompt the user to load one or more pill case containers 102, prompt the user to take a medication pill(s) from a specified one or more of pill cases containers 102, and/or prompt the user to open or close one or more of lids 106. As another example, management engine 220 may notify the user that one or more of lids 106 has been opened prematurely (i.e., prior to a time at which the user is to remove a pill from one of pill case containers 102), and/or notify the user that a pill has not been removed from a pill case container 102 as instructed.

Host 118 (i.e., management engine 220 and/or pill detection and classification engine 222) may populate database 224 with pill weights and images obtained from one or more of a variety of other sources such as, without limitation, pharmaceutical manufacturers, pharmaceutical distributors, retail pharmacies, compounding pharmacies, and pill case platforms 101 (i.e., crowd-sourced pill weights). Host 118 may incorporate external or third party data sets (e.g., an NDC database), explicit datasets (i.e. precise medication weights using calibrated precision equipment), and/or data modifications (i.e. some over the counter medications or supplements have looser manufacturing tolerances).

Pill detection and classification engine 222 classifies weight detection events 114-1 as corresponding to specific pill types based on the weight values of the weight detection events 114-1, pill types contained in patient medication schedule 226, and pill weights of database 224. Host 118 (i.e., management engine 220 and/or Pill detection and classification engine 222), determines whether pill case container 102-1 has been properly loaded and/or unloaded based on the classification and patient medication schedule 226.

Pill detection and classification engine 222 may perform a statistical analysis of pill weights of database 224 to classify weight detection events 114-1 as corresponding to specific pill types. During subsequent loadings (aided loading and/or unaided loading), pill detection and classification engine 222 may incorporate weight detection events 114-1 into a medication classification method to provide a self-reinforcing medication classification method. The medication classification method and/or the self-reinforcing medication classification method may be useful to account for variations (e.g., subtle variations) in weights of a pill type. Such weight variations may be due to natural/inherent variability in characteristics amongst hardware of pill case platforms 101 and/or amongst pill case containers 102 of a pill case platform (e.g., load cell variability and/or ADC bias), environmental factors (e.g., humidity and/or temperature), variability amongst and/or within manufacturers and/or pharmacies, changes in pharmaceutical manufacturing processes, variability in characteristics of user behavior (e.g., loading technique and/or tablet cutting), and/or other factors.

Host 118 (i.e., management engine 220 and/or pill detection and classification engine 222) may maintain database 224 as multiple databases of pill weights, and pill detection and classification engine 222 may maintain a medication classification method and/or a self-reinforcing medication classification method for each database. As an example, host 118 may maintain a unique hardware-specific database of pill weights for each pill case platform 101 and/or for each pill case container 102 of each pill case platform 101. The hardware-specific databases may include records of weight detection events of the respective pill case containers 102 and/or pill case platforms 101. As an example, for each weight detection event 114-1 of pill case container 102-1, pill detection and classification engine 222 may perform a statistical analysis of a hardware-specific database associated with pill case platform 1011 and/or pill case container 102-1 to classify weight detection events 114-1 as corresponding to a specific pill type. During subsequent loadings (aided loading and/or unaided loading), pill detection and classification engine 222 may incorporate weight detection events 114-1 into a classification algorithm of the respective database to provide a self-reinforcing medication classification algorithm. Hardware-specific databases may be useful to accommodate the natural/inherent variability in hardware characteristics described above. Hardware-specific databases may also be useful to accommodate one or more other factors described above.

Alternatively, or additionally, host 118 may maintain unique user-specific databases of pill weights. User-specific databases may be useful to accommodate variability in characteristics of user behavior (e.g., loading technique and/or tablet cutting), and/or other factors (e.g., pharmacy variations and/or environmental conditions).

Alternatively, or additionally, host 118 may maintain a global database of pill weights based on weight detection events of multiple pill case platforms 101. The global database may serve as a crowd-sourced database of pill weights. The global database may be useful to incorporate pharmaceutical variability (e.g., regional differences in pharmaceutical manufacturing facilities and changes in pharmaceutical manufacturing processes), and/or regional variability (e.g., seasonal changes in temperature and humidity) into the medication classification algorithm for use during subsequent loadings (aided loading and/or unaided loading).

Host 118 may perform similar functions with respect to pill case platforms 101-2 through 101-m.

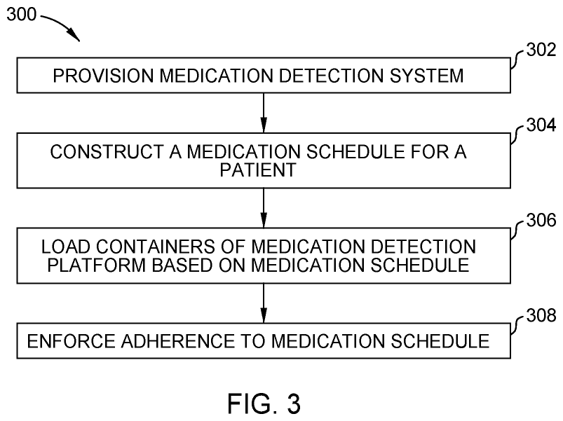

FIG. 3 illustrates a method 300 of ensuring patient adherence to a medication schedule, according to an embodiment. Method 300 is described below with reference to FIGS. 1 and 2, for illustrative purposes. Method 300 is not, however, limited to the examples of FIG. 1 or 2.

At 302, pill case platform 101-1 enters a provisioning mode. Where pill case platform 101-1 is factory-new or a factory-reset, pill case platform 101-1 may enter the provisioning mode when turned on or powered-up.

In the provisioning mode, communication circuitry 250 of pill case platform 101-1 may initiate a point-to-point communication service (e.g., a wireless Bluetooth GATT service) with application 120 of user device 116. Communication circuitry 250 may advertise or broadcast the communication service. Thereafter, application 120 may detect or discover the communication service, and may establish a communication channel or session with pill case platform 101-1 over communication channel 122A. Application 120 and pill case platform 101-1 may exchange cryptographic certificates to authenticate pill case platform 101-1.

After pill case platform 101-1 is authenticated, communication circuitry 250 of pill case platform 101-1 may scan for available wireless access points of communication network 130 (e.g. WiFi, Zigbee, Thread or others). GUI 126 may prompt the user to select a detected access point. GUI 126 may also prompt the user to provide network access credentials such as password (e.g., a WPA2 passkey). GUI 126 may also prompt or permit the user to assign a name to pill case platform 101-1. Thereafter, pill case platform 101-1 may establish a communication session with host 118, via communication channel 122B via the selected wireless access point. If Pill case platform 101-1 loses communication to host 118 via communication channel 122B, pill case platform may also communicate with host 118 by proxy communication via user device 116-1 using communication channel 122A and then from user device 116-1 to host 118 using communication channel 122C.

At 304, management engine 220 enters a configuration mode. Management engine 220 may enter the configuration mode based on user input via GUI 126. In an example, the user may initiate the configuration mode at any time and/or for any reason subsequent to provisioning at 302.

In the configuration mode, management engine 220 may prompt the user (i.e., via GUI 126) to input a list of pill types for patient medication schedule 226. Management engine 220 may permit the user to specify a pill type based on a National Drug Code (NDC) marking and/or a Universal Product Code (UPC) marking. Management engine 220 may permit the user to specify the medication via manual input via GUI 126, and/or via an image capture device (e.g., a camera-based scanner of user device 116-1 and/or other device capable of scanning barcodes). Management engine 220 may permit the user to enter a brand name and/or generic name.

Based on user input, management engine 220 may search database 224 for the pill types identified by the user, and may return a list of pharmaceutical pills and/or over-the-counter pills, or a filtered version of the list, to the user via GUI 126. Management engine 220 may also provide descriptions and/or images of the pill types. GUI 126 may permit the user to select pill types from the list, and may confirm the user selections via GUI 126.

Management engine 220 may also prompt the user to provide a patient dosage schedule (e.g., pill quantity and pill day/time) for each pill type. Management engine 220 may permit the user to specify dosage times in terms of day(s) of the week, time(s) of day, and/or frequency. Management engine 220 may permit the user to specify a dosage frequency in terms of daily, every k days, and/or specific days of the week. Management engine 220 may permit the user to specify a precise dosage time (e.g.,8:00 am), and/or a window of time (e.g., +/−1 hour, or between 7:00 am and 9:00 am). Management engine 220 may permit the user to specify a pill reload time reminder in a similar fashion. Dosage scheduling and reload scheduling are not, however, limited to the foregoing examples. Management engine 220 may construct or populate patient medication schedule 226 for pill case platform 101-1 based on the foregoing configuration parameters received from the user.

At 306, management engine 220 enters a medication loading mode.

Management engine 220 may enter one of multiple user-selectable loading modes, which may include, without limitation, an aided loading mode, an un-aided loading mode, and/or a midweek loading/unloading mode. A midweek loading/unloading mode may be useful if a pill type is added/removed from patient medication schedule 226 midweek (e.g., a doctor makes a change to a patient's medication regimen).

In the aided loading and unloading modes, management engine 220 provides detailed step-by-step instructions for loading and un-loading pills into pill case containers 102 based on patient medication schedule 226, such that each of pill case container 102 contains the appropriate pill types and quantities for a respective day of the week (i.e., one or more of pill case containers 102 may include multiple pills of a given pill type and/or multiple pill types). As an example, and without limitation, management engine 220 may instruct the user to load or unload pills into/from pill case containers 102 in a serial fashion (e.g., one pill case at a time, one pill type at a time). Management engine 220 may initially instruct the user to open one or more of lids 106-1 through 106-n. Management engine 220 may then instruct the user to load or unload a designated number of one or more pill types in/from pill case container 102-1. Thereafter, management engine 220 may instruct the user to load or unload a designated number of one or more pill types in pill case container 102-2, one-at-a time or multiple pills at a time.

Further in the aided loading and unloading modes, pill detection and classification engine 222 may classify/correlate weight detection events 114-1 to pill types of patient medication schedule 226 as the user loads or unload pills into/from pill case containers 102-1. As an example, pill detection and classification engine 222 may determine an expected weight based on specified pill type(s) and quantities and corresponding pill weights from database 224, and may compare the expected weight to weight values of weight detection events 114-1.

If pill detection and classification engine 222 detects an error, pill detection and classification engine 222 or management engine 220 may alert the user via GUI 126, via a signaling device of pill case platform 101-1, and/or other method. For example, and without limitation, management engine 220 may send a push notification to user device 116-1 identifying pills that need to be added or removed from pill case container 102-1. Alternatively, or additionally, management engine 220 may alert the user by activating a signaling device of pill case platform 101-1 and/or a signaling device of pill case container 102-1. A push notification may identify loaded pill case container 102-1 as incorrectly loaded, and may further identify an incorrectly loaded pill type and quantity. The push notification may instruct the user to add or remove a corrective quantity of incorrectly loaded pill types to/from pill case container 102-1. If applicable/appropriate, the message may further instruct the user to move a corrective quantity of incorrectly loaded pill types from pill case container 102-1 to another one of the pill case containers 102 (e.g., from Monday's pill case to Sunday's pill case). After the user corrects the loading error, pill detection and classification engine 222 may re-evaluate weight detection events 114-1. When pill detection and classification engine 222 determines that pill case container 102-1 is properly loaded, management engine 220 may proceed to instruct the user to load or unload pills into/from remaining pill case containers 102-2 through 102-n, in a similar fashion.

In the un-aided loading mode, a user loads pill case containers 102 with quantities of pill types as specified in patient medication schedule 226, without guidance from host 118. The user may place the specified quantities of the specified pill types in pill case containers 102 in any order. When the user indicates that loading of pill case containers 102 is complete (e.g., by closing lids 106-1 to 106-n, and/or by other method), pill detection and classification engine 222 may evaluate weight detection events of pill case containers 102-1 through 102-n based on patient medication schedule 226 and database 224. As an example, for pill case container 102-1, pill detection and classification engine 222 may determine an expected weight based on specified quantities of specified pill types of patient medication schedule 226, and corresponding pill weights from database 224. Pill detection and classification engine 222 may compare the expected weight to weight values of weight detection events 114-1.

If pill detection and classification engine 222 detects a loading error, management engine 220 may alert the user. Management engine 220 may alert the user via GUI 126, via a signaling device of pill case platform 101-1, via a signaling device of one or more incorrectly loaded pill case containers 102, and/or other method, such as described above. The user adds and/or removes pill types and/or quantities in one or more pill case containers 102 as directed by management engine 220 instructions and/or alerts described above. When pill detection and classification engine 222 determines that pill case containers 102-1 through 102-n are properly loaded, management engine 220 may instruct the user to close lids 106-1 through 106-n, if any of lids 106 are open.

At 308, management engine 220 enters a medication adherence mode, in which management engine 220 provides instructions to the user to remove pills from pill case containers 102-1 through 102-n based on patient medication schedule 226. In the medication adherence mode, weight event detector 210 may monitor weights of pill case containers 102 such as described below.

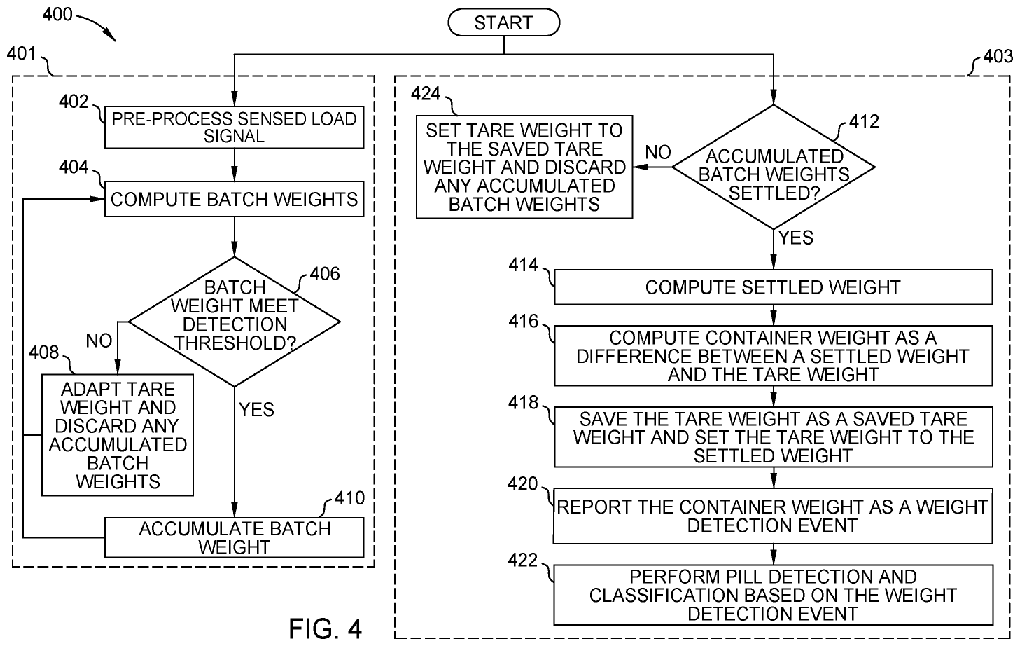

FIG. 4 illustrates a method 400 of determining weight detection events 114-1, according to an embodiment. Method 400 may be performed during aided loading/unloading, unaided loading/unloading, and/or upon completion of unaided loading/unloading. Method 400 may be performed for each pill case container 102 of pill case platforms 101-1 through 101-m, independent of one another. Method 400 is described below with reference to pill case container 102-1. Method 400 is not, however, limited to the example of pill case container 102-1.

In the example of FIG. 4, method 400 includes a thresholding portion 401 and a batch weight processing portion 403. Thresholding portion 401 is described below. Batch weight processing portion 403 is described further below.

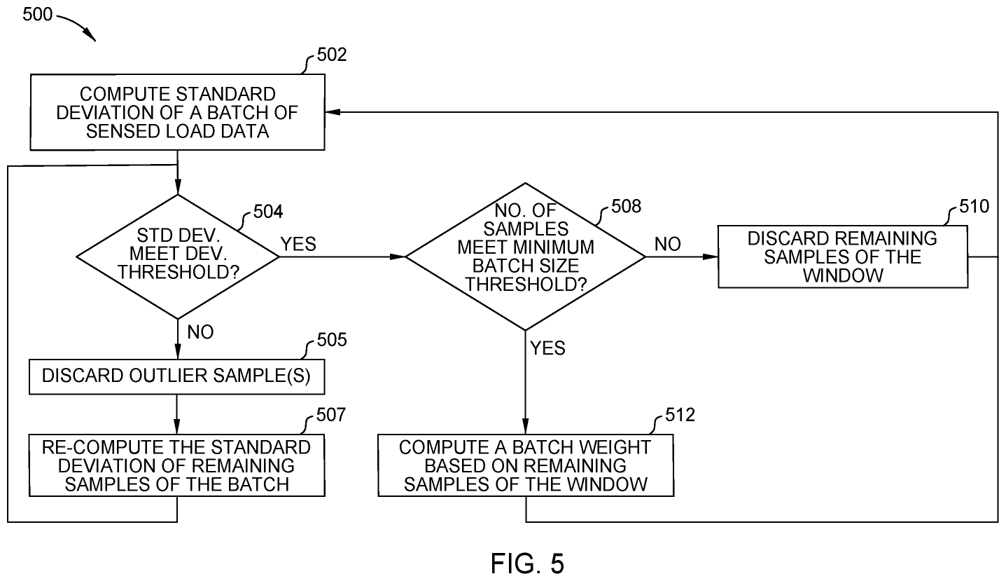

At 402, pre-processor 206 pre-processes sensed load data 204, examples of which are provided further below with reference to FIG. 5.

At 404, pre-processor 206 computes batch weights 208 based on sensed load data 204. Pre-processor 206 may compute batch weights 208 when one or more of lids 106 are open and/or upon another condition(s). Pre-processor 206 may compute batch weights 208 for batches of sensed load data 204 (i.e., samples of sensed load signal 112 within a window of time). Pre-processor 206 may also perform noise reduction and/or filtering, examples of which are provided further below.

At 406, weight event detector 210 compares a batch weight 208 to a detection threshold. The detection threshold may be set to a relatively low value (e.g., a value that is equal to or less than a weight of a lightest pill weight of patient medication schedule 226). A relatively low threshold may be useful to distinguish between batch weights 208 of newly added pills, and batch weights 208 that do not represent newly added pills. As described below, batch weights 208 that do not represent newly added tablets may be used for adaptive taring.

If the batch weight 208 does not meet the detection threshold, processing proceeds to 408, where an adaptive taring engine 214 adapts tare weight 216 based on the batch weight 208. Adaptive taring engine 214 may, for example, accumulate the batch weight 208 with other batch weights 208 of sensed load data 204 that do not meet the detection threshold, and may low-pass filter the accumulated batch weights to provide tare weight 216. Sensed load signal 112-1 may drift over time (i.e., load cell drift) due to temperature and/or other factors. Adapting tare weight 216 based on low-pass filtering of batch weights 208 that do not meet the detection threshold may be useful to compensate for load cell drift.

If the batch weight 208 meets the detection threshold, processing proceeds to 410, where weight event detector 210 accumulates the batch weight 208 with other batch weights 208 of sensed load data 204 that meet the detection threshold at 406.

A batch weight 208 that meets the detection threshold at 406 does not necessarily represent a newly loaded pill. For example, a batch weight 208 may meet the detection threshold due, at least in part, to pressure applied by a user when placing a pill in pill case container 102-1 and/or motion of pill case container 102-1 (e.g., as a user loads a pill into pill case container 102-1, initial batch weighs 208 of sensed load data 204 may spike or fluctuate due to user motion/pressure, but will eventually settle to a relatively steady/stable value).

Thus, at 412, as weight event detector 210 accumulates batch weights 208 of sensed load data 204 that meet the detection threshold, settling detector 212 determines whether the accumulated batch weights 208 have settled. Settling detector 212 may consider, for example and without limitation, median and/or standard deviation values of the accumulated batch weights 208, and/or may low-pass filter the accumulated batch weights 208. If the accumulated batch weights 208 settle, processing proceeds to 414 where weight event detector 210 computes a settled weight based on the accumulated batch weights 208 that meet the detection threshold. Because the underlying accumulated batch weights 208 have settled, the settle weight may be computed with a relatively high degree of confidence.

At 416, weight event detector 210 computes a container weight for pill case container 102-1 based on a difference between the settled weight and tare weight 216.

At 418, adaptive taring engine 214 saves tare weight 216 as a saved tare weight 218, and sets tare weight 216 to the settled weight. In other words, adaptive taring engine 214 dynamically adapts tare weight 216 based on current contents of pill case container 102-1. Thus, going forward, tare weight 216 includes the weight of any pills contained within pill case container 102-1. In this way, if another pill is subsequently added to pill case container 102-1, weight event detector 210 will compute a corresponding container weight that excludes pre-existing contents of pill case container 102-1 (i.e., the container weight will correspond to a newly added pill).

At 420, weight event detector 210 reports the container weight as a weight detection event 114-1. The weight detection event 114-1 may include the container weight and an identifier associated with pill case container 102-1.

At 422, pill detection and classification engine 222 evaluates weight detection event 114-1 to classify/correlate weight detection event 114-1 to a pill type. If weight detection event 114-1 occurs as a user is loading or unloading pill case container 102-1, pill detection and classification engine 222 may evaluate weight detection event 114-1 based on corresponding loading or unloading instructions provided to the user and corresponding pill weights of database 224.

Pill detection and classification engine 222 may classify/correlate weight detection event 114-1 to a single pill of a pill type, a quantity of pills of a pill type, and/or a combination of one or more pills of each of multiple pill types. Pill detection and classification engine 222 may evaluate weight detection event 114-1 based on pill weights of one or more databases 224 (e.g., a hardware-specific pill weight database, a user-specific pill weight database, and/or a global pill weight database). Pill detection and classification engine 222 may evaluate weight detection events 114-1 based on one or more of a variety of methods, which may include a hardware-specific method, a user-specific method, a global method, a proprietary method, and/or other method. In an embodiment, weight detection events 114-1 include container weights computed by weight event detector 210 (e.g., mean weights, container weights, and/or standard deviations), and pill detection and classification engine 222 incorporates these values into a medication classification method to provide a self-reinforcing pill classification method, such as described further above.

Returning to 412, if the accumulated batch weights 208 that meet the detection threshold do not settle, processing proceeds to 424 where adaptive taring engine 214 sets tare weight 216 based on saved tare weight 218 (i.e., reverts tare weight 216 to saved tare weight 218 and resumes adaptive taring function), and discards accumulated batch weights 208 of pill case container 102-1. Reverting tare weight 216 to saved tare weight 218 may be useful for discarding/filtering weights that are not a direct result of a single pill (or multiple pills) being loaded/unloaded (e.g. artificial weight induced by user pressure and/or motion) while continuing to account for load cell drift. As an example, and without limitation, if tare weight 216 is x, an artificial/unintended weight is y, and load cell drift is z, z may be computed based on x+y+Z-y . . . . Otherwise z would be lost during the +y and −y artificial events.

FIG. 5 illustrates a method 500 of pre-processing sensed load data, according to an embodiment (e.g., 402 in FIG. 4). Method 500 is described below with reference to pill case container 102-1. Method 500 is not, however, limited to the example of pill case container 102-1.

At 502, pre-processor 206 computes the standard deviation of a batch of samples sensed load data 114. Pre-processor 206 may compute the standard deviation based on amplitudes of the samples. The batch of samples may include successive samples of sensed load data 114 or sensed load signal 112 within a window of time. The samples may represent amplitudes of sensed load signal 112-1.

At 504, pre-processor 206 compares the standard deviation to a deviation threshold to determine whether the samples within the batch are relatively consistent with one another. If the standard deviation exceeds the deviation threshold, processing proceeds to 506, where pre-processor 206 discards one or more outlier samples of the batch. Pre-processor 206 may compare each sample of the batch to the standard deviation to identify the most egregious outlier(s) to discard.

At 507, pre-processor 206 re-computes the standard deviation of remaining samples of the batch. Processing then returns to 504, where pre-processor 206 compares the re-computed standard deviation to the deviation threshold. Pre-processor 206 may repeat 504, 506, and 507 until a re-computed standard deviation meets the deviation threshold.

When the standard deviation of the original batch of samples, or when a re-computed standard deviation meets the deviation threshold, processing proceeds to 508, where pre-processor 206 compares the number of remaining samples of the batch to a minimum batch size threshold. If the number of samples or remaining samples of the batch does not meet the minimum batch size threshold, processing proceeds to 510, where pre-processor 206 discards the remaining samples of the batch. Processing then returns to 502, where pre-processor 206 evaluates another batch of sensed load data 204.

If the number of remaining samples of the batch meet the minimum batch size threshold at 504, processing proceeds to 512, where pre-processor 206 computes a batch weight 208 based on the remaining samples of the batch. Pre-processor 206 may compute batch weight 208 as an average of the amplitudes of the remaining samples. Processing then returns to 502, where pre-processor 206 evaluates another batch of sensed load data 204.

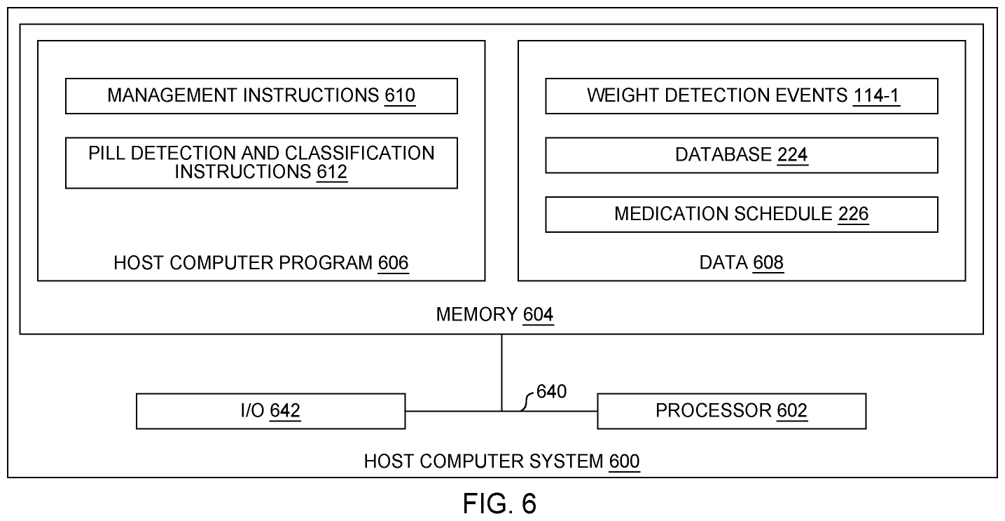

FIG. 6 is a block diagram of host computer system 600, according to an embodiment. Host computer system 600 may represent an example embodiment or implementation of host 118. In the example of FIG. 6, host computer system 600 includes one or more instruction processors, illustrated here as a processor 602, that executes instructions of a host computer program 606 encoded within a non-transitory computer-readable medium, illustrated here as memory 604. Memory 604 further includes data 608, which may be used by processor 602 while executing host computer program 606, and/or generated by processor 602 while executing host computer program 606.

In FIG. 6, host computer program 606 includes management instructions 610 that cause processor 602 to perform management functions, such as described with respect to management engine 220 in one or more examples above. Host computer program 606 further includes pill detection and classification instructions 612 that cause processor 602 to perform pill detection and classification functions, such as described with respect to pill detection and classification engine 222 in one or more examples above.

Host computer system 600 further includes communications infrastructure 640 to communicate amongst devices and/or resources of host computer system 600. Host computer system 600 further includes one or more input/output (I/O) devices and/or controllers (I/O) 642 that interfaces between host computer system 600 with one or more other systems (e.g., pill case platforms 101 and user devices 116), such as described in one or more examples above.

In the preceding, reference is made to embodiments presented in this disclosure. However, the scope of the present disclosure is not limited to specific described embodiments. Instead, any combination of the described features and elements, whether related to different embodiments or not, is contemplated to implement and practice contemplated embodiments. Furthermore, although embodiments disclosed herein may achieve advantages over other possible solutions or over the prior art, whether or not a particular advantage is achieved by a given embodiment is not limiting of the scope of the present disclosure. Thus, the preceding aspects, features, embodiments and advantages are merely illustrative and are not considered elements or limitations of the appended claims except where explicitly recited in a claim(s).

As will be appreciated by one skilled in the art, the embodiments disclosed herein may be embodied as a system, method or computer program product.

Accordingly, aspects may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, aspects may take the form of a computer program product embodied in one or more computer readable medium(s) having computer readable program code embodied thereon.

Any combination of one or more computer readable medium(s) may be utilized. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium is any tangible medium that can contain, or store a program for use by or in connection with an instruction execution system, apparatus or device.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium may be transmitted using any appropriate medium, including but not limited to wireless, wireline, optical fiber cable, RF, etc., or any suitable combination of the foregoing.

Computer program code for carrying out operations for aspects of the present disclosure may be written in any combination of one or more programming languages, including an object oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Aspects of the present disclosure are described below with reference to flowchart illustrations and/or block diagrams of methods, apparatus (systems) and computer program products according to embodiments presented in this disclosure. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

The flowchart and block diagrams in the Figures illustrate the architecture, functionality, and operation of possible implementations of systems, methods, and computer program products according to various examples of the present invention. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

While the foregoing is directed to specific examples, other and further examples may be devised without departing from the basic scope thereof, and the scope thereof is determined by the claims that follow.

What is claimed is:

1. A system, comprising:
a pill case platform comprising a pill case container, a load cell, and circuitry configured to determine a container weight of the pill case container based on an output of the load cell; and
a host device configured to determine whether the pill case container contains specified quantities of multiple pill types of a patient medication schedule based on the container weight and a database of pill types and associated pill weights.

2. The system of claim 1, wherein the host device is further configured to determine a corrective quantity of one or more of the multiple pill types if the host device determines that the pill case container does not contain the specified quantities of the multiple pill types of the patient medication schedule.

3. The system of claim 1, wherein the host device is further configured to determine whether the pill case container contains an incorrect pill type based on the container weight, the patient medication schedule, and the database of pill types and associated pill weights.

4. The system of claim 1, wherein the database comprises multiple pill weights for one or more of the multiple pill types of the patient medication schedule, and wherein the host device is further configured to:
determine whether the pill case container contains the specified quantities of the multiple pill types of the patient medication schedule based further on a statistic determined based on the multiple pill weights.

5. The system of claim 4, wherein the multiple pill weights are based on one or more of:
prior container weights of the pill case platform and the patient medication schedule; and
container weights of other pill case platforms and patient medication schedules associated with the other pill case platforms.

6. The system of claim 4, wherein the statistic comprises one or more of a standard deviation of the multiple pill weights and a mean of the multiple pill weights.

7. The system of claim 1, wherein the host device is further configured to determine whether the pill case container contains the specified quantities of the multiple pill types of the patient medication schedule by:
computing an expected weight of the pill case container based on the specified quantities of the multiple pill types of the patient medication schedule and corresponding pill weights of the database;
comparing the container weight to the expected weight; and
determining that the pill case container contains the specified quantities of the multiple pill types of the patient medication schedule if the container weight matches the expected weight within a statistical measure of certainty.

8. The system of claim 7, wherein the host device is further configured to determine a corrective quantity of one or more of the multiple pill types of the patient medication schedule if the container weight does not match the expected weight within the statistical measure of certainty, including to:
compute alternative expected weights of the pill case container based on various quantities of the multiple pill types of the patent medication schedule and the corresponding pill weights of the database;
compare the container weight to the alternative expected weights; and
determine that the pill case container contains an incorrect quantity of one or more of the multiple pill types of the patient medication schedule if the container weight matches one of the alternative expected weights within the statistical measure of certainty.

9. The system of claim 8, wherein the host device is further configured to:
determine the corrective quantity based on the alternative expected weight that matches the expected weight within the statistical measure of certainty.

10. The system of claim 8, wherein the host device is further configured to:
determine that the pill case container contains an incorrect pill type if the container weight does not match any of the alternative expected weights within the statistical measure of certainty.

11. The system of claim 1, wherein the host device is further configured to send an alert to the pill container platform if the host device determines that the pill case container does not contain the specified quantities of the multiple pill types of the patient medication schedule.

12. The system of claim 1, wherein the host device is further configured to provide a corrective instruction to a user device associated with the pill case platform if the host device determines that the pill case container does not contain the specified quantities of the multiple pill types of the patient medication schedule, and wherein the corrective instruction comprises one or more of:
an instruction to add a corrective quantity of one or more of the multiple pill types of the patient medication schedule to the pill case container;
an instruction to remove a corrective quantity of one or more of the multiple pill types of the patient medication schedule from the pill case container; and
an instruction to remove a corrective quantity of an incorrect pill type from the pill case container.

13. The system of claim 12, wherein the host device is further configured to:
provide an image of the one or more pill types identified in the corrective instruction, to the user device.

14. The system of claim 1, wherein the host device is further configured to:
provide an instruction for loading the pill case container with the specified quantities of the multiple pill types of the patient medication schedule, to a user device associated with the pill case platform; and
provide images of the multiple pill types with the instruction.

15. The system of claim 1, wherein the pill case platform comprises multiple pill case containers, and wherein the host device is further configured to:
provide instructions for loading respective ones of the pill case containers based on the patient medication schedule, serially.

16. The system of claim 1, wherein the pill case platform comprises multiple pill case containers, and wherein the host device is further configured to:
provide instructions for loading respective ones of the multiple pill types of the patient medication schedule, to a user device associated with the pill case platform.

17. The system of claim 1, wherein the pill case platform comprises multiple pill case containers, and wherein the host device is further configured to:
determine whether the pill case containers contain respective specified quantities of the multiple pill types of the patient medication schedule based on respective container weights, the database of pill types and associated pill weights, and an indication that the pill cases are loaded in accordance with the pill schedule;
wherein the indication comprises one or more of,
an indication from the pill case platform that lids of the pill case containers are closed, and
an indication from a user device that the pill cases are loaded in accordance with the pill schedule.

18. A non-transitory computer readable medium encoded with a computer program that comprises instructions to cause a processor to:
associate a pill case platform with a patient and with a user device, wherein the pill container platform comprises multiple pill case containers;
receive a medication schedule of the patient from the user device;
determine specified quantities of multiple pill types of the patient medication schedule, for each of the pill case containers, based on the patient medication schedule;
receive indications of container weights from the pill case platform; and
determine whether the pill case containers contain the specified quantities of the multiple pill types of the patient medication schedule based on the container weights and a database of pill weights and associated pill types.

19. The non-transitory computer readable medium of claim 18, further comprising instructions to cause the processor to:
send a confirmation to one or more of the user device and the pill case platform if the pill case containers contain the specified quantities of the multiple pill types of the patient medication schedule; and
determine a determine a corrective quantity of one or more of the multiple pill types for one or more of the pill case containers and provide a corrective instruction to the user device if one or more of the pill case containers do not contain the specified quantities of the multiple pill types of the patient medication schedule.

20. The non-transitory computer readable medium of claim 18, further comprising instructions to cause the processor to:
provide instructions for loading the specified quantities of the multiple pill types of the patient medication schedule to the user device.

21. The non-transitory computer readable medium of claim 18, further comprising instructions to cause the processor to:
determine whether the pill case container contains an incorrect pill type based on the container weight, the patient medication schedule, and the database of pill types and associated pill weights.
